(19) United States
(12) Patent Application Publication
(10) Pub. No.: US 2022/0370258 A1
(43) Pub. Date: Nov. 24, 2022

(54) Catamenial support accessory

(71) Applicant: Ceketha E. Yarbrough, Brandon, FL (US)

(72) Inventor: Ceketha E. Yarbrough, Brandon, FL (US)

(21) Appl. No.: 17/326,173

(22) Filed: May 20, 2021

(51) Int. Cl. A61F 13/475; A61F 13/472; A61F 13/84; A61L 15/40

(52) U.S. Cl. CPC A61F 13/4758; A61F 13/472; A61F 13/84; A61L 15/40

(57) ABSTRACT

A soft fibrous highly absorbent non-woven resilient cotton roll formed in a pliable symmetrical cylindrical solid structure which allows for conformity and comfort used for catamenial purposes in concave region of the gluteus maximus.

### CROSS-REFERENCE TO RELATED APPLICATION

This application claims priority to U.S. Provisional Patent Application No. 62/704,662 filed [May 20, 2020], the entirety of which is incorporated by reference.

### FIELD OF THE INVENTION

The present invention relates generally to feminine hygiene care products, and, more particularly, relates to the use of highly absorbent cotton rolls of fibrous material for catamenial flow issues, especially as it relates to Menorrhagia, and it can be used for hemorrhoids.

### BACKGROUND OF THE INVENTION

Menorrhagia is defined as excessive menstrual bleeding and more than 1 in five women around the world suffer with it. Yet, half of the women in the United States who experience menorrhagia don't seek treatment. This information is based on a study done by Gynecare a division of Ethicon, Inc a Johnson & Johnson company, published in 2002 and relates to my first provisional patent filed in 1999 application No. 60/392,045 now expired.

The focus on menstrual products has been from the prospective of the center parts only of the Vagina on a woman's body. Of course, that makes sense because that is where the flow commences during her menses and the market is saturated with products such as menstrual pads/sanitary napkins and tampons of all sizes, to provide coverage for her while she is flowing on her period, yet overflow accidents continue to happen, especially as it relates to a woman lying down on her back, this usually can lead to overnight overflows onto the bedding.

The known prior art such as US patent numbers 3871378 & 3994298 & 4389211, these well-known catamenial bandages, has not completely covered women in all areas of the vaginal region that are susceptible to the menses blood flow path in channels and directions that are completely missed during the menstrual cycle. The blood flow path can escape the coverages of menstrual pads, sanitary napkins and tampons, of all sizes, that are in the market today.

Reference patent number U.S. Pat. No. 8,216,202 describes why and how women can experience leakage while wearing a tampon using a term called “early failure” of a tampon, stated in the Background section of that Invention, 2^{nd} paragraph.

Therefore, a need exists to overcome the problems with the prior art as discussed above. This invention provides additional coverage and absorbency in a new position of placement to help protect women during their menstrual flow particularly in the region of the gluteus maximus, with a prior art, reference here; U.S. Pat. No. 4,705,514, as a highly absorbent cotton roll also known as dental rolls.

### SUMMARY OF THE INVENTION

In accordance with some embodiments of the inventive disclosure, there is provided a method of preventing menstrual absorbency device leakage that includes providing a menstrual pad that is configured to be placed adjacent a vaginal opening to receive menstrual fluids, and providing a roll of a non-woven elongated fibrous material formed in a solid cylinder at a rear portion of the menstrual pad. The method further includes placing the roll between the buttocks of a user in close proximity to the bony prominence of the sacrum & coccyx region of the body in the concave area for external use.

In accordance with a further feature, providing roll of non-woven elongated fibrous material comprises providing a roll of cellulosic fibers including perforations therein to facilitate a flow of fluids into the roll from the menstrual pad.

In accordance with a further feature, placing the roll comprising placing the roll adjacent a perineal region of the user.

In accordance with a further feature, the method further includes attaching the roll to the rear end of the menstrual pad.

In accordance with some embodiments of the inventive disclosure, there is provided a device for absorbing menstrual fluids that includes a menstrual pad configured to be placed adjacent a vaginal opening to receive menstrual fluids, and a roll of a non-woven elongated fibrous material formed in a solid cylinder at a rear end of the menstrual pad. The roll is configured to be placed between the buttocks of a user, and to absorb menstrual fluid from the pad.

In accordance with a further feature, roll is comprised of cotton.

In accordance with a further feature, the roll is comprised of cellulosic fibers including perforations therein to facilitate a flow of fluids into the roll from the blood flow path of the buttocks channel along the fatty tissue of the gluteus maximus and can absorb overflow of the menstrual pad.

In accordance with a further feature, the roll has a length of three to nine inches, and a diameter of three eights of an inch to one half inch.

Although the invention is illustrated and described herein as embodied in a catamenial support device and method of use, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

“In the description of the embodiments of the present invention, unless otherwise specified, azimuth or positional relationships indicated by terms such as “up”, “down”, “left”, “right”, “inside”, “outside”, “front”, “back”, “head”, “tail” and so on, are azimuth or positional relationships based on the drawings, which are only to facilitate description of the embodiments of the present invention and simplify the description, but not to indicate or imply that the devices or components must have a specific azimuth, or be constructed or operated in the specific azimuth, which thus cannot be understood as a limitation to the embodiments of the present invention. Furthermore, terms such as “first”, “second”, “third” and so on are only used for descriptive purposes, and cannot be construed as indicating or implying relative importance.

In the description of the embodiments of the present invention, it should be noted that, unless otherwise clearly defined and limited, terms such as “installed”, “coupled”, “connected” should be broadly interpreted, for example, it may be fixedly connected, or may be detachably connected, or integrally connected; it may be mechanically connected, or may be electrically connected; it may be directly connected, or may be indirectly connected via an intermediate medium. As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. Those skilled in the art can understand the specific meanings of the above-mentioned terms in the embodiments of the present invention according to the specific circumstances.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1A is a perspective view of the highly absorbent roll/tail product material for catamenial purposes to be used as a support accessory to the menstrual pad.

FIG. 1B is a perspective view of a menstrual pad shown with the product roll/tail attached as a tail, which can be used as one unit as the roll/tail piece has no adhesive.

FIG. 2A is a perspective view depicting the placement of the support accessory roll/tail in between the channel of the buttocks of a user.

FIG. 2B is a transparent view depicting the placement of the support accessory roll/tail in between the channel of the buttocks of a user.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The principal reason of this invention is to provide supplemental absorbent coverage while wearing a menstrual pad or tampon, to help fully protect women during their menstrual cycles from a rear angle especially those women who suffer with menorrhagia, excessive heavy bleeding. This product can also be used on hemorrhoids for both women and men, although the main focus is for catamenial benefits.

A full detailed description of the cylindrical symmetrical roll comprising of non-woven fibrous and highly absorbent resilient cellulosic materials is given in prior art, U.S. Pat. No. 2,608,901 in accordance with Goldman and 4705514 Ralph Barnard. This patent describes the highly absorbent fibrous materials used in the inventive embodiments, but for a different function of the body, a new method of use for catamenial purposes in between the gluteus maximus channel instead of for use in the mouth. The sizes for my invention vary from about 3″-9″ inches long and between ⅜″-½″ inch in diameter.

The foregoing US patent description of materials can be used with a new method and in a new bodily zone for a new purpose is nonobvious based on today's market in 2021 of feminine care products. There is not even a category for catamenial support accessories such as a kind of absorbent material to be used in conjunction with the principal products that are in the marketplace now. The traditional conventional menstrual pads and tampon are the only choices that women have in today's market, since the 1930's, and it is not nor has been sufficient for a protecting a woman from all angles.

That flow that flows along the curvy region of the fatty tissue of the gluteus maximus (buttocks), rear end, my invention works best in this area and especially when lying down. It also works great absorbing that “flash splash” velocity that she didn't know was coming, rather she's standing or sitting. Simply put when a woman has her menses that is the time that she wants as much coverage as necessary to feel secure and to prevent overflow accidents onto garments and furnishings.

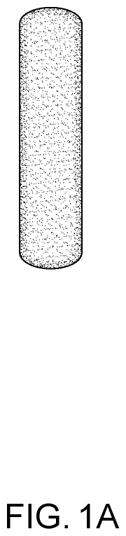

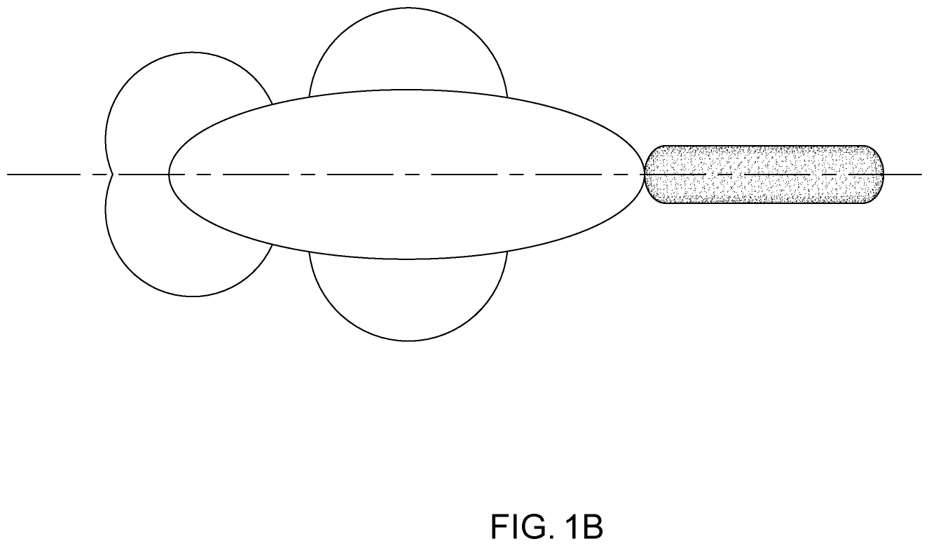

FIG. 1A shows a perspective view of the highly absorbent plug product material for catamenial purposes to be used as a supplemental accessory to the menstrual pad. It is a highly absorbent nonwoven solid fibrous material.

FIG. 1B is a perspective view of a menstrual pad shown with the product plug/tail attached which can be used as one unit as the plug/tail piece has no adhesive. A catamenial bandage for menstrual flow purposes in conjunction with the cylindrical symmetrical cotton roll/tail, shown as a possible attachment.

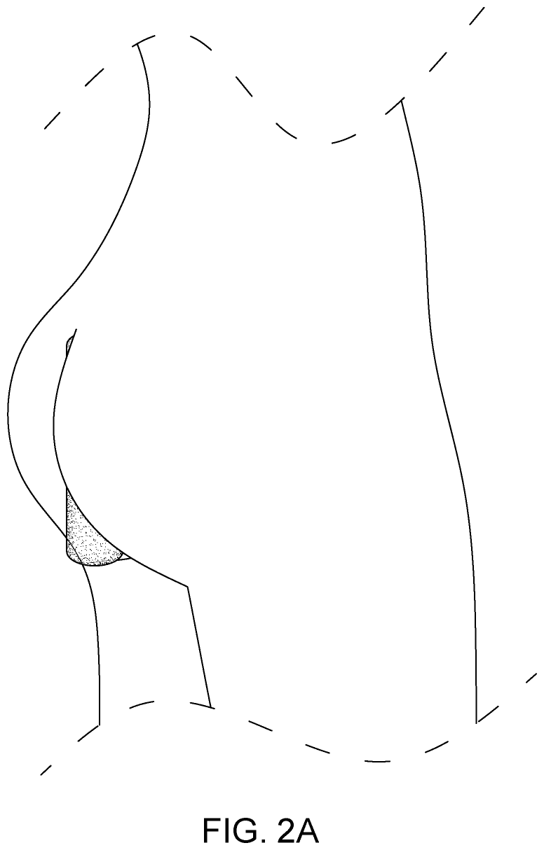

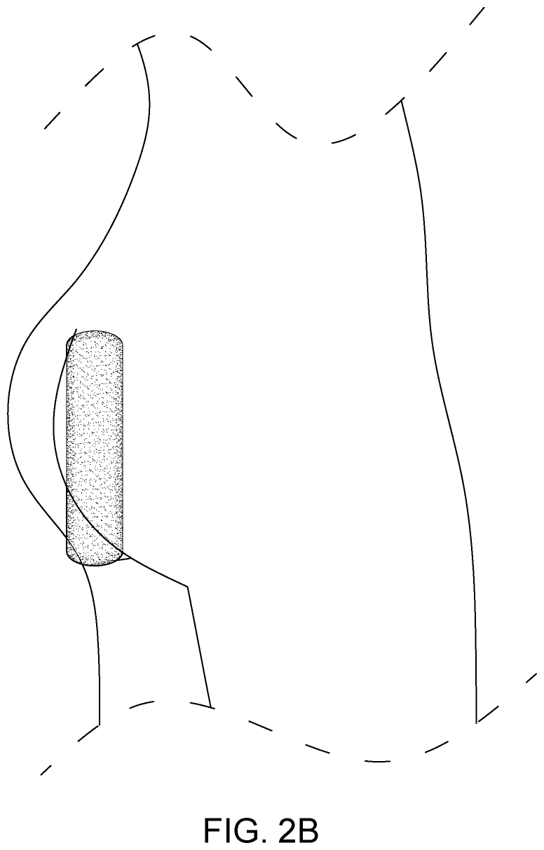

FIG. 2A is a perspective view depicting the placement of the supplemental accessory roll/plug in between the channel of the buttocks of a user.

FIG. 2B is a transparent view depicting the placement of the supplemental accessory roll/plug in between the channel of the buttocks of a user.

A catamenial device has been disclosed as an accessory to support current market products, (menstrual/sanitary pads and tampons) which reduces or helps eliminate leakage of the menses by providing an absorbent cotton roll/tail in conjunction with said products, that can be held between the user's buttocks.

The claims appended hereto are meant to cover all modifications and changes within the scope and spirit of the present invention.

What is claimed is:

1. A method of preventing menstrual leakage by an absorbency device, comprising:
providing a menstrual pad that is configured to be placed adjacent a vaginal opening to receive menstrual fluids,
providing a roll of a non-woven elongated fibrous material formed in a solid cylinder at a rear end of the menstrual pad; and
placing the roll between the buttocks of a user in close proximity to the bony prominence of the sacrum & coccyx region of the body in the concave area for external use.

2. The method claim 1 wherein providing roll of non-woven elongated fibrous material comprises providing a roll of cellulosic fibers including perforations therein to facilitate a flow of fluids into the roll from the menstrual flow and leakage from the menstrual pad.

3. The method of claim 1 wherein placing the roll comprising placing the roll adjacent a perineal region of the user's buttocks.

4. The method of claim 1 further comprising, attaching the roll to the rear end of the menstrual pad.

5. A device for absorbing menstrual fluids, comprising:
a menstrual pad configured to be placed adjacent a vaginal opening to receive menstrual fluids; and
a roll of a non-woven elongated fibrous material formed in a solid cylinder at a rear end of the menstrual pad;
wherein the roll is configured to be placed between the buttocks of a user, and to absorb menstrual fluid from the pad.

6. The device of claim 5, wherein roll is comprised of cotton.

7. The device of claim 5, wherein the roll is comprised of cellulosic fibers including perforations therein to facilitate a flow of fluids into the roll from the menstrual blood path flow.

8. The device of claim 1, wherein the roll has a length of three to nine inches, and a diameter of three eights of an inch to one half inch.
